SEQUENCE LISTING (2 sequences)

SEQ ID NO: 1 - DNA, 327 bp, Mus musculus
  source 1..327: mol_type = unassigned DNA
gaaattgtgctcacccagtctccaaccaccatggctgcatctcccggggagaagatcactatcacctgcagtgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtataccgctcacgttcggtgctgggaccaagctggagctgaaacgg

SEQ ID NO: 2 - DNA, 360 bp, Mus musculus
  source 1..360: mol_type = unassigned DNA
gatgtgcagcttcaggagtcaggacctgacctggtgaaaccttctcagtcactttcactcacctgcactgtcactggctactccatcaccagtggttatagctggcactggatccggcagtttccaggaaacaaactggaatggatgggctacatacactacagtggtagcactaactacaacccatctctcaaaagtcgaatctctatcactcgagacacatccaagaaccagttcttcctgcagttgaattctgtgactactgaggacacagccacatattactgtgcaagagaactacggctacggtacttcgatgtctggggcgcagggaccacggtcaccgtctcctcagcctcc

(19) United States
(12) Patent Application Publication
(10) Pub. No.: US 2025/0155457 A1
(43) Pub. Date: May 15, 2025

(54) METHOD FOR ANALYZING DEPENDENT HEPARIN PLATELET ACTIVATION

(71) Applicants: DIAGNOSTICA STAGO, ASNIERES SUR SEINE (FR); UNIVERSITÉ DE TOURS, TOURS (FR); CENTRE HOSPITALIER RÉGIONAL UNIVERSITAIRE DE TOURS, TOURS (FR)

(72) Inventors: Yves Jean Hubert GRUEL, TOURS (FR); Jérôme ROLLIN, ROCHECORBON (FR); Noémie Aurélie Ludivine CHARUEL, ASNIERES-SUR-SEINE (FR); Claire POUPLARD, TOURS (FR)

(21) Appl. No.: 18/833,154

(22) PCT Filed: Jan. 27, 2023
(86) PCT No.: PCT/EP2023/052029

§ 371 Date: Jul. 25, 2024

(30) Foreign Application Priority Data
Jan. 28, 2022 (FR) 2200734

(51) Int. Cl. G01N 33/86

(52) U.S. Cl. CPC G01N 33/86; G01N 2800/222

(57) ABSTRACT

The present invention relates to a method for analyzing platelet activation induced by antibodies directed against the polyanion-modified platelet factor 4 (PF4), preferably directed against the PF4/heparin complex, as well as to a method of in vitro diagnosis of heparin-induced thrombocytopenia or of a diagnosis of exclusion of a heparin-induced thrombocytopenia in a patient, comprising specific steps.

The present invention relates to a method for analyzing platelet activation induced by antibodies directed against the polyanion-modified platelet factor 4 (PF4), preferably against the PF4/heparin complex, and to a method of in vitro diagnosis of heparin-induced thrombocytopenia or of a diagnosis of exclusion of a heparin-induced thrombocytopenia in a patient, comprising specific steps.

Heparin-induced thrombocytopenia (HIT) is a severe complication of heparin therapy. HIT is the consequence of an atypical immune response characterized by the synthesis of IgG class antibodies specifically directed against the polyanion-modified platelet factor 4 (PF4), e.g. heparin (H). The anti-polyanion-modified PF4 antibodies activate platelets via the FcγRIIa receptor.

Heparin is widely used in hemodialysis, in cardiac surgery in particular with Extracorporeal circulation (ECC), in extracorporeal membrane oxygenation (ECMO) procedures and in intensive care. In common practice, HIT is frequently suspected in hospitalized patients treated with heparin, but the diagnosis is confirmed only in approximately 1-3% of patients receiving unfractionated heparin (UFH) therapy, and more rarely in the patients treated with low molecular weight heparin (LMWH).

It is important to note that among patients treated with heparin, some develop anti-polyanion-modified PF4 antibodies that do not induce platelet activation. Same are not pathogenic and hence not responsible for a HIT. Such is in particular the case during ECMO or CEC procedures, during which nearly 50% of patients develop anti-polyanion-modified PF4 antibodies, but the antibodies are only apt to activate platelets in 2 to 3% of cases.

HIT is actually considered a clinical and biological syndrome, the complete diagnosis of which is based on:
1. The occurrence of one or a plurality of clinical events associated with HIT (thrombocytopenia, thrombosis, etc.), and
2. The demonstration, in the plasma or the serum of the patient, of polyanion-modified anti-PF4 antibodies and apt to activate platelets in vitro in the presence of heparin.

Two types of tests are used for the biological diagnosis of a HIT:
immunological tests, which detect the presence of anti-polyanion-modified PF4 antibodies, regardless of the ability thereof to activate cells; and
functional tests, which demonstrate heparin-dependent activation of platelets by the antibodies. In practice, platelet activation is demonstrated in the presence of therapeutic concentrations of heparin (i.e. comprised between 0.1 and 1 IU/ml heparin), whereas the activation is completely inhibited by a high concentration of heparin (i.e. comprised between 10 and 100 IU/ml).

The main advantage of immunological tests is the high sensitivity thereof (with an excellent negative predictive value), explaining that a negative result serves in a large majority of cases to exclude the illness. Nevertheless, because of the insufficient positive predictive value thereof, a positive result should be associated, in the vast majority of cases, with a functional test to confirm or not confirm the pathogenicity of antibodies and hence the diagnosis of HIT. The combination of the two types of tests is necessary to confirm an HIT, or at least to considerably improve the specificity of biological diagnosis.

To date, all functional tests available require fresh platelets or blood from healthy donors. The effect of patients' plasma or serum on platelet activation is studied in the presence of heparin. Some tests also add exogenous PF4 to make more sensitive, the detection of platelet activation induced by pathogenic antibodies.

The most powerful tests are the radiolabeled serotonin release assay (SRA) and the heparin-induced platelet aggregation assay (HIPA), both performed with fresh washed platelets obtained from one or a plurality of healthy donors. The sensitivity and specificity of SRA and HIPA are greater than 95%.

Other functional tests that also use platelets from healthy subjects are also available but have poorer performance. To date, two commercial tests use the flow cytometry technique: Emo-Test HIT Confirm® (5-Diagnostics, Basel, Switzerland) and HITAlert™ KIT (IQ products, DL Groningen, the Netherlands).

The performances of functional tests available today are variable, giving heterogeneous results due to the use of different methodologies, to the variability of washed platelets from healthy donors, and also to different positivity criteria to confirm HIT.

In addition, many studies have demonstrated a variable reactivity of platelets to HIT antibodies, which implies that it is often necessary to perform the tests with platelets from a plurality of healthy subjects before concluding reliably to a negative result for a patient.

The use of platelets from healthy subjects actually has a plurality of pitfalls:
the prior agreement of healthy subjects must be obtained before sampling;
the great difficulty of accessing healthy donors;
donor selection, due to high variability in platelet response from one subject to another; and
the washing step which can have an impact on the activity of the platelets.

Finally, the vast majority of tests available today are carried out after a platelet isolation step. However, HIT results from multicellular activation induced by pathogenic anti-PF4/H antibodies. Thereby, monocytes and neutrophils are also stimulated by the antibodies and can potentiate platelet activation. Currently, only one functional test for the diagnosis of HIT is performed on whole blood (HIMEA: Heparin-induced Multiple Electrode Aggregometry), which also requires a sample from a healthy subject.

There is thus a very strong need, more particularly for haemostasis laboratories, for a sensitive and specific functional test, which does not require the sampling of healthy donors and which also does not require a platelet isolation step.

The lack of standardization of functional diagnostic tests for HIT is also a major pitfall. Haemostasis laboratories need a functional test the interpretation of the results of which is standardized and the result turnaround time is quite short (such as e.g. less than 24 h to 48 h outside the weekend).

The present invention makes it possible to meet such expectations.

A further subject matter of the present invention is thereby a method for analyzing platelet activation in a patient suspected of having thrombocytopenia induced by anti-polyanion-modified PF4 antibodies, more particularly antibodies to the heparin-modified PF4.

The present invention also relates to a method for the in vitro diagnosis of a HIT or for confirming the diagnosis of a HIT in a patient suspected of having a HIT.

The invention also provides a method for in vitro diagnosis of the exclusion of HIT in a patient suspected of having HIT.

According to the first embodiment of the invention, the subject matter of the present invention is a method for analyzing heparin-dependent platelet activation in a patient suspected of having thrombocytopenia induced by anti-polyanion-modified PF4 antibodies, in particular antibodies directed against the heparin-modified PF4 factor, comprising the following steps:
1) the preparation of a series of tests necessary for validation and standardization, hereinafter referred to as the Validation/Standardization series (VS), said Validation/Standardization series comprising:
   mixing a sample of whole blood from the patient, diluted in an acceptable buffer, in the presence of exogenous platelet factor 4 (PF4), and a monoclonal antibody specifically directed against the platelet factor 4 modified by a polyanion, preferably by heparin, or one of the fragments thereof, in order to obtain a mixture 1a;
   at least one mixture of a sample of whole blood from said patient, diluted in an acceptable buffer, with exogenous PF4, a monoclonal antibody specifically directed against polyanion-modified PF4, or a fragment thereof, and heparin in a concentration comprised between 0.01 and 1 IU/ml, in order to obtain at least one mixture 1 b;
   at least one mixture of a sample of whole blood from said patient, diluted in an acceptable buffer, with exogenous PF4, a monoclonal antibody specifically directed against polyanion-modified PF4, or a fragment thereof, and heparin in a concentration comprised between 10 and 200 IU/ml, in order to obtain at least one mixture 1c;
2) the preparation of a Patient Test (PT) series, said Patient Test series comprising:
   mixing a sample of whole blood from said patient, diluted in an acceptable buffer, with a platelet activator or a strong agonist, to obtain a mixture Ago;
   mixing a sample of whole blood from said patient, diluted in an acceptable buffer, to obtain the mixture Basal;
   mixing a sample of whole blood from said patient, diluted in an acceptable buffer, with PF4 to obtain a mixture 2a;
   at least one mixing of a sample of whole blood from said patient, diluted in an acceptable buffer, with PF4 and heparin at a concentration equal to the concentration used for mixture 1b, in order to obtain a mixture 2b;
   at least one mixing of a sample of whole blood from said patient, diluted in an acceptable buffer, with PF4 and heparin at a concentration equal to the concentration used for mixture 1c, in order to obtain a mixture 2c;
3) the incubation of all mixtures of the VS and TP series and the subsequent labeling of the platelet population and the specific labeling of activated platelets in said population in each mixture; and
4) the analysis of all mixtures obtained in step 3).

The method according to the invention is an in vitro method.

Preferably, step 4) of analyzing all the mixtures obtained in step 3) comprises measuring the specific labeling intensity of activated platelets in the platelet population of each mixture of the VS series (1a to 1c) and of the TP series (Ago, Basal, and 2a to 2c).

Preferably, the method of the invention is a flow cytometry analysis method.

Preferably, the method of the invention comprises the steps 1) to 4) set out above, as well as the following additional step 5), following step 4):
5) the validation of the VS series.

According to the invention, it is considered that the VS series is validated if one observes:
an activation of platelets in the sample of said series induced by an antibody or antibody fragment directed specifically against the polyanion-modified PF4, in the absence of heparin and/or in the presence of low concentrations of heparin, and
an inhibition of the activation in the presence of high concentrations of heparin.

Preferably, step 5) consists in the validation the VS series by the calculation of the activation ratio of the series and the calculation of the inhibition ratio of the same series.

For the purposes of the present invention, a heparin-dependent platelet activation refers to a platelet activation induced by anti-polyanion-modified PF4 antibodies (or antibody fragments), or antibodies (or antibody fragments) mimicking anti-polyanion-modified PF4 antibodies, which is observed in the absence of heparin and/or in the presence of low concentrations of heparin, and which is inhibited in the presence of high concentrations of heparin.

The term “low concentration of heparin” means a concentration typically comprised between 0.01 and 1 IU/ml.

The term “high concentration of heparin” means a concentration typically comprised between 10 and 200 IU/ml.

Following step 5), the method of the invention preferably comprises a step 5′) of calculation of the antibody-dependent platelet activation level (called APA) of the mixtures Ago, 2a, 2b and 2c of the TP series.

Advantageously, the method of the invention is implemented for the diagnosis of exclusion of a HIT, or the confirmation of a diagnosis of HIT in a patient suspected of having the illness.

In such case, the method of the invention comprises the steps 1) to 5′) set out above, as well as a step of interpreting the results obtained on the TP series in order to establish a diagnosis of a HIT or an exclusion of HIT.

According to a particular embodiment of said application, the method of the invention comprises the steps 1) to 5′) set out above, as well as a step 6), wherein the step 6) is as follows:
6) interpretation (preferably comparison) of antibody-dependent platelet activation (APA) rates calculated in each TP series mixture for establishing a diagnosis of HIT or of an exclusion of HIT.

The method according to the invention, the steps and the operating conditions thereof will now be discussed in detail according to the indications hereinbelow.

The method uses samples of whole blood from a patient. “Whole blood” means a blood sample comprising plasma and the formed elements (red blood cells, white blood cells and platelets and derived microparticles).

The use of whole blood from the patient serves to dispense with the variability of response to HIT antibodies from platelets from healthy donors. Thereby, the real activator character of the patient's antibodies is directly evaluated on the patient's own platelets.

Moreover, the use of whole blood of the patient brings the benefit of the multicellular cooperation often involved during a HIT.

In fine, the consequences of heparin-dependent and multicellular activation induced by anti-polyanion-modified PF4 antibodies in the patient's own platelets are evaluated.

Preferably, each sample is obtained from blood samples collected in the presence of a compound preventing the blood from coagulating. The compound commonly used is sodium citrate, but other compounds such as hirudin or citric acid-trisodium citrate-glucose (ACD) can also be used for such purpose. Such compounds have long been known in the prior art to serve for the study of platelet functions.

Preferably, the total volume sample of whole blood from the patient is comprised between 500 μl and 2 ml.

The sample of whole blood may be diluted in any physiologically acceptable buffer. Physiological saline (i.e. aqueous NaCl 9 g/l solution) is preferably used, but other buffers such as e.g. the PBS buffer with different compositions (e.g. NaN₃ 10× or Citrate NaN₃ 10×) can also be used for the dilution of the blood sample.

All the samples of whole blood of the patient used in the method according to the invention are used undiluted or diluted up to 1/10th, preferably diluted in the proportion 1/2 to 1/10^{th}, and preferably a quarter, in physiological saline. Such a dilution makes it possible to use thrombopenic blood (i.e. wherein the quantity of platelets less than 150 Giga/I).

Typically, all the mixtures of steps 1) and 2) (VS and TP series) of the method of the invention are obtained with the same buffer.

### Composition and Preparation of the Validation/Standardization Series (VS, Step 1)

The VS series comprises at least 3 mixtures:
the mixture 1a, which is the mixture of a sample of whole blood from the patient, diluted in an acceptable buffer, with exogenous PF4 and a monoclonal antibody specifically directed against polyanion-modified PF4, or a fragment thereof;
at least one mixture 1 b, which corresponds to the mixture of a sample of whole blood from the patient, diluted in an acceptable buffer, with exogenous PF4, a monoclonal antibody specifically directed against polyanion-modified PF4, or a fragment thereof, and heparin in a concentration comprised between 0.01 and 1 IU/ml; and
the mixture 1c, which corresponds to the mixture of a sample of whole blood from said patient, diluted in an acceptable buffer, with exogenous PF4, a monoclonal antibody specifically directed against polyanion-modified PF4, or one of the fragments thereof, and heparin at a concentration comprised between 10 and 200 IU/ml.

Preferably, each of the mixtures 1a to 1c has a volume comprised between 20 and 200 μl.

In practice, the above antibody perfectly mimics the effects of HIT antibodies developed by patients. It is used as an internal positive quality control and thereby serves for the technical validation of the test, as well as a standardized expression of the results, which contributes to the innovative character of the invention.

Preferably, the preparation of the VS series comprises the preparation of at least 4 mixtures:
the mixture of a sample of whole blood from said patient, diluted in an acceptable buffer, with exogenous PF4 and a monoclonal antibody specifically directed against polyanion-modified PF4, or one of the fragments thereof, to obtain mixture 1a;
at least two mixtures of samples of whole blood from said patient, diluted in an acceptable buffer, with exogenous PF4, a monoclonal antibody specifically directed against polyanion-modified PF4, or a fragment thereof, and heparin at a concentration comprised between 0.01 and 1 IU/ml, the concentration of heparin being different for each mixture, so as to obtain at least two mixtures 1b; and
the mixture of a sample of whole blood from the patient, diluted in an acceptable buffer, with exogenous PF4, a monoclonal antibody specifically directed against polyanion-modified PF4, or a fragment thereof, and heparin in a concentration comprised between 10 and 200 IU/ml, in order to obtain a mixture 1c.

Preferentially, two mixtures 1b (i.e. mixtures 1b′ and 1b″) are carried out, on the one hand with a concentration of heparin of comprised between 0.01 and 0.1 IU/ml for the first mixture 1b′, and on the other hand comprised between 0.2 and 1 IU/ml for the second mixture 1b″. More preferentially, the two mixtures 1b are carried out with a concentration of heparin comprised between 0.02 and 0.08 IU/ml, preferably about 0.05 IU/ml, for the first mixture 1b′; and with a concentration of heparin comprised between 0.3 and 0.8 IU/ml, preferably about 0.5 IU/ml, for the second mixture 1b″.

The mixture 1c is obtained more preferentially with a concentration of heparin comprised between 50 and 150 IU/ml, preferably between 80 and 120 IU/ml, preferably about 100 IU/ml.

Mixtures 1 b (1b′ and 1b″) mimic the effects of low concentrations of heparin, while mixture 1c mimics the effects of high concentrations of heparin.

### Monoclonal Antibody Specifically Directed Against Polyanion-Modified PF4

“Monoclonal antibody specifically directed against polyanion-modified PF4, or a fragment thereof” refers to an antibody or to an active fragment thereof (I) which binds to polyanion-modified PF4, and which does not bind, or binds insignificantly, to PF4 alone, and (ii) which induces platelet activation and aggregation in the absence and/or presence of low concentrations of heparin, and which is inhibited in the presence of high concentrations of heparin. Preferably, such an antibody or an active fragment thereof (I) binds to the PF4/heparin complex (“PF4/H complex”), and does not bind, or binds insignificantly, to PF4 alone, and (ii) induces platelet activation and aggregation in the absence and/or presence of low concentrations of heparin, and the action of which is inhibited in the presence of high concentrations of heparin.

As will appear hereinafter in the description of the invention, the low concentrations of heparin in the presence of which such an antibody activates platelets are generally comprised between 0.01 and 1 IU/ml. The high concentrations in the presence of which an inhibition of the activating effect of said antibody is observed are generally comprised between 10 and 200 IU/ml, typically on the order of 100 IU/ml.

Preferably, the antibody is a whole antibody. Preferably, the antibody is an IgG, preferably an IgG1. The antibody may be chimeric, humanized, or human. Preferentially, the antibody comprises a human Fc.

“Antibody” refers to a tetramer made up of two heavy chains of 50-70 kDa each (called H chains for Heavy) and two light chains of about 25 kDa each (called L chains for Light), linked by intra- and inter-catenary disulfide bridges and identical to each other. The tetramer has at least two variable regions at the N-terminal end of each chain (called VL for light chains and VH for heavy chains) and a constant region at the C-terminal end, consisting of a single domain called CL for the light chain and three or four domains for the heavy chain called CH1, CH2, CH3 and, if appropriate, CH4. The region that determines the specificity of the antibody for the antigen is carried by the variable parts VH and VL, said parts being responsible for the recognition of the antigen. In each VH and VL variable region, three loops are combined to form an antigen binding site. Each loop is called a complementarity-determining region (or CDR).

The assembly of the chains that make up an antibody serves to define a characteristic three-dimensional Y-shaped structure, where
the base of Y corresponds to the constant region Fc, or Fc fragment: the base is recognized by the proteins of the complement and the Fc receptors in order to mediate the effector functions of the antibody, and
the ends of the arms of the Y correspond to the respective assembly of the VL variable region of a light chain and the VH variable region of a heavy chain, said ends forming the region Fab and determining the specificity of the antibody for the antigen.

The Fab fragment has the same affinity for the antigen as the complete antibody. The Fab fragment is formed of the whole light chain (VL+CL) and of a part of the heavy chain (VH+CH1). Same is monovalent.

Preferably, the monoclonal antibody (or one of the fragments thereof) directed specifically against the polyanion-modified PF4, and preferably against the PF4/H complex, comprises the VH and VL sequences encoded by the following nucleic acid sequences:

Nucleic acid sequence encoding the VL variable
region (VJ):
(SEQ ID NO: 1)
GAAATTGTGCTCACCCAGTCTCCAACCACCATGGCTGCATCTCCCGGGG
AGAAGATCACTATCACCTGCAGTGCCAGCTCAAGTATAAGTTCCAATTA
CTTGCATTGGTATCAGCAGAAGCCAGGATTCTCCCCTAAACTCTTGATT
TATAGGACATCCAATCTGGCTTCTGGAGTCCCAGCTCGCTTCAGTGGCA
GTGGGTCTGGGACCTCTTACTCTCTCACAATTGGCACCATGGAGGCTGA
AGATGTTGCCACTTACTACTGCCAGCAGGGTAGTAGTATACCGCTCACG
TTCGGTGCTGGGACCAAGCTGGAGCTGAAACGG
Nucleic acid sequence encoding the VH variable
region (VDJ):
(SEQ ID NO: 2)
GATGTGCAGCTTCAGGAGTCAGGACCTGACCTGGTGAAACCTTCTCAGT
CACTTTCACTCACCTGCACTGTCACTGGCTACTCCATCACCAGTGGTTA
TAGCTGGCACTGGATCCGGCAGTTTCCAGGAAACAAACTGGAATGGATG
GGCTACATACACTACAGTGGTAGCACTAACTACAACCCATCTCTCAAAA
GTCGAATCTCTATCACTCGAGACACATCCAAGAACCAGTTCTTCCTGCA
GTTGAATTCTGTGACTACTGAGGACACAGCCACATATTACTGTGCAAGA
GAACTACGGCTACGGTACTTCGATGTCTGGGGCGCAGGGACCACGGTCA
CCGTCTCCTCAGCCTCC

Preferably, the monoclonal antibody (or one of the fragments thereof) directed specifically against the polyanion-modified PF4, and preferably against the PF4/H complex according to the invention comprises:
as VH variable region, the amino acid sequence encoded by SEQ ID NO:2, and
as VL variable region, the amino acid sequence encoded by SEQ ID NO:1.

Such an antibody is in particular the antibody 5B9, described more particularly in Kizlik-Masson et al. (Journal of Thrombosis and Haemostasis, 15: 2065-2075, 2017, 5B9, a monoclonal antiplatelet factor 4/heparin IgG with a human Fc fragment that mimics heparin-induced thrombocytopenia antibodies).

As indicated hereinabove, such an antibody is used in the method according to the invention as a positive internal quality control, which allows a standardized expression of the results taking into account the platelet activation measured with the antibody.

### Composition and Preparation of the Patient Test Series (PT, Step 2)

The Patient Test (TP) series comprises 5 mixtures, 2 of which are additional to the Validation/Standardization series: the Ago mixture and the Basal mixture.

Mixtures 2a, 2b (optionally 2b′ and 2b″) and 2c are similar to mixtures 1a, 1b (optionally 1b′ and 1b″) and 1c respectively (i.e. same quantities, same ingredients and same volumes). However, the 5 mixtures of the Patient Test (PT) series do not include the addition of monoclonal antibodies specifically directed against polyanion-modified PF4, or any of the fragments thereof.

The Patient Test series assesses the ability of polyanion-modified anti-PF4 antibodies in the patient's blood to induce (1) platelet activation in the absence of heparin and/or in the presence of low concentrations of heparin, and (ii) platelet inactivation in the presence of high concentrations of heparin.

The presence of a platelet activator or strong agonist in the Ago mixture is intended to activate the entire platelet population and is a non-specific platelet activation control to define the positivity threshold of the platelet activation marker.

The strong agonist is known in the prior art. Among the different strong agonists, mention can be made of TRAP (thrombin receptor activator peptides, with 6 or 14 amino acids), calcium ionophore A23187 (chemical formula hereinafter), arachidonic acid, adenosine diphosphate (ADP), or thrombin and the derivatives thereof. Preferably, two types of strong agonists are used according to the final labeling of platelet activation: preferably, the strong agonist is chosen from TRAP (thrombin receptor activator peptides, with 6 or 14 amino acids) or calcium ionophore A23187, the chemical formula of which is given below:

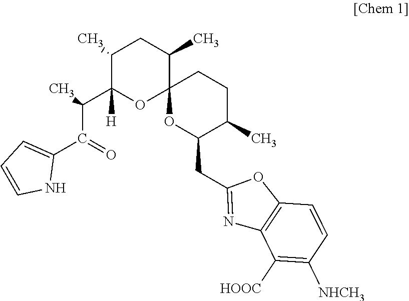 [SMILES: CNc1ccc2oc(C[C@H]3O[C@@]4(CC[C@H]3C)O[C@H]([C@H](C)C(=O)c3ccc[nH]3)[C@H](C)C[C@H]4C)nc2c1C(=O)O]

The Basal mixture is a control made in order to have the basal level of platelet activation. The buffer of the Basal mixture is a buffer known from the prior art. Preferably, the buffer of the Basal mixture is a saline buffer such as a PBS buffer, a PBS-BSA 1% buffer, a Tyrodes HEPES-BSA 1% buffer, a PBS-BSA-NaN₃ 10×buffer, a PBS-citrate-NaN₃ 10×buffer or a PBS 10×NaN₃ buffer.

The two controls (ago and Basal) serve to position the positivity thresholds of the markers used to determine the antibody-dependent platelet activation level.

Preferably, the preparation of the Patient Test series comprises:
mixing a sample of whole blood from said patient, diluted in an acceptable buffer, with the strong agonist to obtain the Ago mixture;
mixing a sample of whole blood from said patient, diluted in an acceptable buffer, with buffer, in order to obtain a Basal mixture;
mixing a sample of whole blood from said patient, diluted in an acceptable buffer, with PF4, to obtain mixture 2a;
at least one mixing of a sample of whole blood from said patient, diluted in an acceptable buffer, with PF4 and heparin at a concentration equal to the concentration used for mixture 1b, in order to obtain a mixture 2b. Preferably, at least two mixtures of a sample of whole blood from said patient, diluted in an acceptable buffer, with PF4 and heparin to obtain at least two 2b mixtures (2b′ and 2b″), the concentration of heparin of each 2b mixture being equal to the concentration of each 1 b mixture;
the mixing of a sample of whole blood from said patient, diluted in an acceptable buffer, with PF4 and heparin at a concentration equal to the concentration used for mixture 1c, in order to obtain a mixture 2c.

Preferably, steps 1) and 2) of the method according to the invention are carried out after 4 h, preferably after 12 h, preferably after 24 h after obtaining the samples of whole blood from the patient. Preferably, steps 1) and 2) of the method according to the invention are carried out no later than 72 h, preferably no later than 48 h after obtaining the samples of whole bloods from the patient.

The method according to the invention thereby serves to generate heparin-dependent platelet activation, directly assessed in the whole blood of the patient, on the patient's own platelets. The above imparts the invention a considerable advantage over the tests proposed in the prior art, since the invention dispenses with the constraints associated with the supply of batches of platelets from healthy donors, a supply hitherto required by the tests prior to the invention.

### Incubation and Labeling (Step 3)

Once each series has been prepared, all the mixtures of steps 1) and 2) are incubated and then the activated platelets are labeled.

Incubation is typically carried out for a period of 10 minutes to 4 hours at a temperature comprised between 18° C. and 37° C.

Preferably, platelet labeling is performed using at least one marker specific to the platelet population and at least one marker specific to the activation state of platelets. Platelet labeling may be performed using at least one marker specific to the platelet population and possibly two markers specific to the activation state of platelets.

Such labeling with a specific marker of the activation state of platelets consists in revealing and quantifying the presence of any compound or molecule the expression, synthesis or the activation of which is induced when the platelets are in an activated state and the level of synthesis or activation of which can be used to assess the level of platelet activation.

Preferably, the compound is chosen from molecules or antigens expressed on the surface of platelets, such as P-selectin (or CD62P), phosphatidylserine (PS), LAMP-3 (or CD63) protein, ADP, ATP, phosphorylated proteins or combinations thereof. Preferably, the compounds used for labeling are P-selectin (or CD62P) and phosphatidylserine (PS).

In order to identify and/or quantify this compound(s), antibodies or antibody fragments directed to the latter, or molecules apt to specifically recognize the selected compound(s) and demonstrating the recognition, are preferably used.

Typically, CD62P (or P-selectin) is a relevant candidate for assessing platelet activation. CD62P is a protein contained in platelet alpha granules, which is expressed on the surface of platelets during platelet activation.

An anti-CD62P antibody is thus preferentially used to label the expression of CD62P.

In addition to CD62P, or alternatively to CD62P, the Annexin V (or AnxV) protein can also demonstrate platelet activation. Same has a strong affinity for phosphatidylserine (PS), which, initially located in the cytoplasmic sheet of the resting cell, is exposed to the surface of the activated platelets.

When Annexin V is used as a marker, all mixtures used in the method according to the invention (steps 1 and 2) are obtained by adding a thrombin inhibitor. Such a thrombin inhibitor is preferably a direct thrombin inhibitor, preferably hirudin or argatroban. The addition of the inhibitor prevents the formation of thrombin, and secondarily of fibrin. Moreover, when Annexin V is used as a marker, all the mixtures used in the method according to the invention (steps 1 and 2) are carried out by adding a calcium buffer. Indeed, the addition of a calcium buffer is essential for the exposure of phosphatidylserines and hence for labeling with Annexin V.

In parallel, a specific labeling of the platelet population is used, targeting a compound expressed at the surface by all the platelets. Such a compound is, e.g., the CD41 marker, the CD61 marker or the combination of the CD41/CD61 markers. Preferably, an anti-CD41 antibody is used for the specific labeling of platelets in the sample.

Preferably, the labels used are coupled with fluorochromes, e.g. fluorescent proteins. Such fluorochromes are well known in the prior art, and are e.g. allophycocyanin (APC), phycoerythrin (PE), phycoerythrin (PE)-Cy5 or Cy7, PerCP, PerCP-Cy5.5, fluorescein (FITC) or else Alexa Fluor dyes.

The use of two platelet activation controls (e.g. CD62P and PS) can optimize the performance of the method, more particularly in terms of specificity and sensitivity.

Preferably, before the analysis of all the mixtures, preferentially on a flow cytometer, a large volume of acceptable buffer, typically comprised between 100 μl and 5 ml, is added to each mixture. The last step slows down the activation of platelets induced during steps 1 and 2, before analysis. The buffer is preferably the same as the buffer used specifically for labeling P-selectin or phosphatidylserines with Annexin V.

Analysis of Incubated Mixtures with Markers and Calculation of Platelet Activation Level from Measurement of the Specific Labeling Intensity of Activated Platelets in the Platelet Population of Each Mixture (Steps 4 and 5′)

Once the platelets have been marked, the latter are analyzed, preferably by flow cytometry. Indeed, the flow cytometry technique is a particularly effective tool for assessing platelet activation in the presence of a reduced number of cellular elements. The above is of major interest in the present case, since the blood of patients with a suspicion of HIT is most often thrombopenic.

Thereby, according to such preferred embodiment, step 4) of analyzing all the mixtures obtained in step 3) comprises measuring the level of expression of CD62P and/or the exposure of phosphatidylserine to the platelet surface (via labeling with Annexin V) of each mixture.

Optionally, the method further comprises a step 5′) of calculation of the antibody-dependent platelet activation level (APA) in each of the mixtures.

According to a preferred embodiment, the platelet analysis of the mixtures of each of the 2 Validation/Standardization and Patient Test series (step 4) as well as the calculation of the antibody-dependent platelet activation (APA) level of the Ago, 2a, 2b and 2c mixtures of the TP series (step 5′) are carried out according to a fenestration strategy presented in the example, the description of which is given in more detail hereinafter.

Briefly, the proposed fenestration strategy includes the following steps:
a) a first sorting is carried out on the basis of the measurement of the intensity of labeling of a specific compound of the platelet population to select only the platelets from the other elements of the blood of each mixture, on the peak of a first histogram;
b) in the selected platelet population, the median labeling intensity of a compound expressed by activated platelets (i.e. marker specific to state of activation of platelets), e.g. CD62P and/or PS via labeling with annexin V, is measured for all mixtures in each series on the peak of a second histogram;
c) a threshold is determined for distinguishing non-activated platelets from activated platelets on a third histogram, placed at the intersection between the peak obtained with the Ago mixture (i.e. state of maximal activation of platelets) in the previous step b) and the peak obtained with the Basal mixture (i.e. basal state of platelet activation); and
d) after repositioning the threshold on the second histogram, the percentage of activated platelets doubly marked (i.e. cellular events to the right of the threshold) is calculated for each of the mixtures of each of the 2 series.

According to a preferred mode of expression of the results, the median total labeling intensity of the compound expressed by the activated platelets (i.e. specific marker of the activation state of the platelets) is calculated during step b). Since the labeling is preferably carried out with fluorescent markers, the median fluorescence intensity (MFI) is thus measured.

In such case, for each of the conditions, the results are expressed by multiplying the percentage (%) of cells doubly positive for the marker of the platelet population and for the marker specific for the platelet activation state (e.g. CD41+/CD62P+), by the total MFI of the specific marker of platelet activation state in the platelet population (i.e. MFI of CD62P/CD41+total labeling) (%×MFI). The results are expressed in arbitrary units (AU).

### Technical Validation of the Validation/Standardization Series (Step 5)

Preferably, the method of the invention comprises steps 1) to 5′) set out hereinabove.

According to the invention, the VS series is considered to be validated if one observes:
an activation of platelets in the sample of the series induced by an antibody or antibody fragment directed specifically against the polyanion-modified PF4 in the absence of heparin and/or in the presence of low concentrations of heparin, and
an inhibition of the activation in the presence of high concentrations of heparin.

Preferably, step 5) consists in validating the VS series by the calculation of the activation ratio of the series and the inhibition ratio of the same series. Thereby, according to said embodiment, two criteria are taken into account to technically validate the Validation/Standardization series:

In the VS series, activation should first be observed with the addition of the monoclonal antibody specifically directed against the polyanion-modified PF4, or a fragment thereof, preferably the antibody 5B9, and PF4 in the absence and/or presence of low concentrations of heparin. Thereby, the activation ratio of the VS series is calculated between the Basal mixture in the absence of heparin and of PF4, and the maximum platelet activation obtained in the presence of low concentrations of heparin tested, indicated as mixtures 1 b. The VS series activation ratio is calculated with the following formula:

$$VS series activation ratio = \frac{UA max ( mixture 1 b )}{UA Basal mixture} \qquad [ Math 1 ]$$

Secondly, the heparin-dependence of platelet activation induced by the monoclonal antibody directed specifically against the polyanion-modified PF4, or one of the fragments thereof, preferably the antibody 5B9, is assessed by calculating the inhibition ratio in the presence of the high concentration of heparin tested. The inhibition ratio of the VS series is calculated with the following formula:

$$VS series inhibition ratio = \frac{UA max ( mixture 1 b ) - UA mixture 1 c}{UA max ( mixture 1 b )} \qquad [ Math 2 ]$$

The Validation/Standardization series is validated only if the two previous criteria are met:
Activation ratio VS series threshold value, said threshold value being defined from the mean obtained with sample population and by excluding outliers; and
VS series inhibition ratio greater than or equal to a threshold value, said threshold value being comprised between 0.3 and 0.7, preferably comprised between 0.4 and 0.6, preferably about 0.5.

Preferably, the positivity threshold for the VS activation ratio is at least 5 for the CD62P protocol and at least 4 for the Annexin V protocol.

### Interpretation of the Results of the Patient Test Series for the Diagnosis of Heparin-Induced Thrombocytopenia or for an Exclusion Diagnosis (Steps 5′ and 6)

The validated results of the Validation/Standardization series are used to calculate the antibody-dependent platelet activation level (referred to as APA) for the Basal, 2a, 2b and 2c mixtures of the Patient Test series (step 5′). Said ratio, expressed herein as a percentage, is calculated from the raw results (%×MFI) expressed in AU obtained on the two series by means of the following formula:

$$A P A = 100 \times \frac{UA sample - UA Basal mixture}{UA max ( mixture 1 b ) - UA Basal mixture} \qquad [ Math 3 ]$$

The calculation makes it possible to dispense with a variable reactivity of platelets from one patient to another and from one manipulation to another by standardizing the expression of the results obtained on the Patient Test series.

In parallel, it is also possible, if the maximum APA is higher than the defined threshold, to calculate the percentage inhibition of the TP series according to the following formula:

$$\% T P series inhibition = 100 \times \frac{A P A max ( mixture 2 b ) - A P A mixture 2 c}{A P A max ( mixture 2 b )} \qquad [ Math 4 ]$$

The percentages of activation and inhibition thereby calculated are compared with threshold values of positivity and inhibition established from cohorts of positive and negative control samples, the threshold values being a function of the marker considered and of the expression level thereof on the platelet surface.

According to the results provided by such embodiment, the invention therefore serves to diagnose a HIT in a patient or to exclude a diagnosis of HIT in a patient suspected of having the illness. Preferably, a diagnosis of HIT exclusion is established when no platelet activation is observed in the presence of low concentrations of heparin on the patient's TP series. Preferably, a diagnosis of HIT in a patient suspected of having the illness is confirmed when a platelet activation of the patient's TP series is observed in the presence of low concentrations of heparin and an inhibition of platelet activation is observed in the presence of high concentrations of heparin.

The analysis results thereby obtained are interpreted as follows (step 6):
I/a positive diagnosis of HIT is confirmed when the following two conditions are met for the Patient Test series:
   a high platelet activation (value greater than or equal to the positivity threshold) should be observed in the presence of low concentrations of heparin (mixture 2b); and
   inhibition of the platelet activation (value greater than or equal to the inhibition threshold) should be observed in the presence of the high concentration of heparin tested (mixture 2c). The second condition serves to verify the dependence on heparin of platelet activation induced by anti-polyanion-modified PF4 antibodies present in the patient's blood.
II/a diagnosis of HIT exclusion is established in the following two cases:
   if the APA is below the positivity threshold in the presence of low concentrations of heparin (mixture 2b); or
   if the APA is above the positivity threshold, but the percentage inhibition is below the negativity threshold.
III/The test is considered questionable if the APA is greater than or equal to the positivity threshold in the presence of low concentrations of heparin (mixture 2b), but the percentage inhibition at the high concentration of heparin (mixture 2c) is in the gray zone, i.e. comprised between the negativity threshold and the positivity threshold. In such case, the test should preferably be repeated on a new sample.

When the method is used with CD62P labeling or with annexin V, the results are interpreted as follows:

### For the CD62P Protocol:

I/The test is considered positive if the APA is greater than or equal to the threshold defined in the presence of low concentrations of heparin (mixture 2b) and the percentage inhibition in the presence of high concentration of heparin (mixture 2c) is greater than or equal to 50%.

The APA positivity threshold will be defined on a population of n patients undergoing heparin therapy with thrombocytopenia or a drop in platelet counts greater than or equal to 40% after initiation of heparin therapy. Two different methods can be used:
The positivity threshold of APA can be defined using an ROC curve, preferring a specificity greater than 95%. For this reason, patients with a negative ELISA and a negative SRA are considered not to have the illness, while patients with a positive ELISA and a positive SRA are considered to have the illness. Such method serves to define the positivity threshold of the APA max at 20% antibody-dependent platelet activation.
The APA positivity threshold can also be defined by the mean and standard deviation of APA values measured in a population of patients with suspected HIT with negative ELISA and negative SRA.

II/the test is considered negative in the following two cases:
If APA is below the defined threshold in the presence of low concentrations of heparin (mixture 2b); or
If the APA is above the threshold for low concentrations of heparin (mixture 2b), but the percentage inhibition is below the negative threshold.

III/The test is considered questionable if the APA is greater than or equal to the threshold defined in the presence of low concentrations of heparin (mixture 2b) but the percentage inhibition at the high concentration of heparin (mixture 2c) is comprised between 30 and 50%. In such case, the test should preferably be repeated on a new sample.

FOR the Annexin V protocol:

I/The test is considered positive if the APA is greater than or equal to the threshold defined in the presence of low concentrations of heparin (mixture 2b) and the percentage inhibition in the presence of high concentration of heparin (mixture 2c) is greater than or equal to 50%.

The APA positivity threshold will be defined on a population of n patients undergoing heparin therapy with thrombocytopenia or a drop in platelet counts greater than or equal to 40% after initiation of heparin therapy. Two different methods can be used:
The positivity threshold of APA can be defined using an ROC curve, preferring a specificity greater than 95%. For this reason, patients with ELISA negative and SRA negative are considered not to have the illness, while patients with ELISA positive and SRA positive are considered to have the illness. Such method serves to define the positivity threshold of the APA max at 27% antibody-dependent platelet activation.
The APA positivity threshold can also be defined by the mean and standard deviation of APA values measured in a population of patients with suspected HIT with negative ELISA and negative SRA.

II/the test is considered negative in the following two cases:
If APA is below the defined threshold in the presence of low concentrations of heparin (mixture 2b); or
If the APA is above the threshold for low concentrations of heparin (mixture 2b) the percentage inhibition is below the negative threshold.

III/The test is considered questionable if the APA is greater than or equal to the threshold defined in the presence of low concentrations of heparin (mixture 2b) but the percentage inhibition at the high concentration of heparin (mixture 2c) is comprised between 30 and 50%. In such case, the test should preferably be repeated on a new sample.

The invention is now illustrated with the following example, which example describes an embodiment of the invention by flow cytometry with the markers CD62P and Annexin V, according to a fenestration strategy and a method of interpretation of the results as indicated hereinabove for the establishment of a HIT exclusion diagnosis or a HIT confirmation diagnosis.

### EXAMPLE: IMPLEMENTATION OF THE METHOD ACCORDING TO THE INVENTION

### 1/Reagents Used:

TABLE 1

| Name | Supplier | Composition |
|---|---|---|
| Physiological saline | Aguettant | NaCl 9 g/l. |
| | Laboratory | |
| Antibody 5B9 | Diagnostica Stago | Anti-PF4/H monoclonal |
| | | antibody lyophilized in |
| | | Hepes-Trehalose-BSA |
| | | buffer |
| TRAP-6 | Agro-Bio | |
| TRAP-14 | Polypeptide | |
| | Laboratories | |
| Sodium heparin | PANPHARMA | |
| PF4 | Diagnostica Stago | |
| PBS | Immuno Concepts | |
| Calcium ionophore | Sigma | |
| A23187 | | |
| Calcium buffer | | 1.4M of NaCl, 25 mM |
| | | of CaCl₂, 0.1M of |
| | | HEPES, pH 7.4 |
| Arganova | Aguettant | Argatroban |
| | Laboratory | |
| Hirudin | Diagnostica Stago | |
| Anti-CD41-APC antibodies | Miltenyi | |
| Anti-CD41-PE antibodies | BioCytex | |
| Anti-CD62P-PE antibodies | BioCytex | |
| Annexin V-FITC | Miltenyi Biotech | |

### 2/Test Description (Method According to the Invention):

The test according to the invention is carried out with the sampled whole blood of the patient, on sodium citrate (3.2%, 1:9, 0.109 mmol/I) and diluted in physiological saline (NaCl 9 g/I).

From the patient's blood, two sets of tests are performed:
The Validation/Standardization series is carried out after adding the chimeric monoclonal antibody 5B9, which mimics the HIT antibodies developed by the patients, to the whole blood of the patient. Such series serves:
   to validate the technique by evaluating the ability of the patient's platelets to be activated by the monoclonal antibody 5B9 in the presence of low concentrations of heparin; and
   to standardize the expression of the results of the Patient Test series which is carried out in parallel;
and the Patient Test series serves to assess the ability of anti-PF4/H antibodies present in the patient's blood to induce platelet activation in the presence of low and high concentrations of heparin.

For a better understanding of the examples, it is specified that in the following Tables 2 to 14, the tubes 1 and 7 of the first columns correspond to the mixtures 1a and 2a, respectively, of the Validation/Standardization (VS) and Patient Test (TP) series, the tubes 2-3 and 8-9 correspond to the mixtures 1b and 2b, the tubes 4 and 10 correspond to the mixtures 1c and 2c, the tube 5 corresponds to the Ago mixture and the tube 6 corresponds to the Basal mixture.

### 3/Experimental Procedures:

Labeling of the expression of P-selectin (CD62P):

A first Validation/Standardization series is carried out with the patient's blood diluted (dilution 1/4) after addition of monoclonal antibody 5B9 and incubated with exogenous platelet factor 4 (PF4) (10 μg/mL) and different concentrations of heparin (0, 0.05, 0.5 and 100 IU/mL) (VS series, step 1).

A second series of tests (Patient Test) is carried out under the same conditions but without any addition of the antibody 5B9 (TP series, step 2). During the TP series, a non-specific platelet activation control (Ago mixture) is performed with a powerful agonist (TRAP, 50 μm). A control in the presence of PBS saline buffer (Basal mixture) is also performed in order to have the basal level of platelet activation of the patient. The two controls serve to position the positivity thresholds of the fluorescence markers used.

After incubation, the activated platelets are labeled with anti-CD41-APC and anti-CD62P-PE labeled antibodies (step 3). The use of anti-CD41 antibody makes possible a specific labeling of platelets in the sample, whereas the anti-CD62P antibody is a recognized marker for assessing platelet activation.

The two series of tests are then analyzed by flow cytometry (step 4).

Labeling of Phosphatidylserine Exposure with Annexin V:

A first Validation/Standardization series is carried out with the patient's blood diluted (dilution 1/4) after addition of the monoclonal antibody 5B9 and incubated with exogenous platelet factor 4 (PF4) (10 μg/ml), different concentrations of heparin (0; 0.05; 0.5 and 100 IU/ml), a thrombin inhibitor (hirudin or argatroban), TRAP (100 μM) and a calcium buffer (VS series, step 1).

A second series of tests is carried out under the same conditions but in the absence of the monoclonal antibody 5B9 (TP series, step 2). A non-specific platelet activation control (Ago mixture) is performed with a potent agonist (Calcium ionophore A23187, 5 μM). A control in the presence of PBS saline buffer (Basal mixture) is also carried out in order to have the basal level of platelet activation. The two controls serve to position the positivity thresholds of the fluorescence markers used.

After incubation, the antibody labeled anti-CD41-APC and Annexin V-FITC are added (step 3). Annexin V demonstrates exposure of phospatidylserine on the surface of activated platelets.

The two series of tests are then analyzed by flow cytometry (step 4).

An example of an experimental design performed for CD62P or Annexin V is shown in Table

TABLE 2

| Tube | Composition | SERIES |
|---|---|---|
| 1 | Diluted blood + 5B9 + PF4 | Validation/ |
| | (= mixture 1a) | Standardization |
| 2 | Diluted blood + 5B9 + PF4 + heparin 0.05 IU/ml | |
| | (= first mixture 1b′) | |
| 3 | Diluted blood + 5B9 + PF4 + heparin 0.5 IU/ml | |
| | (= second mixture 1b″) | |
| 4 | Diluted blood + 5B9 + PF4 + heparin 100 IU/ml | |
| | (= mixture 1c) | |
| 5 | Diluted blood + strong agonist (TRAP or Ca | Patient Test |
| | ionophore) (= ago mix) | |
| 6 | Diluted blood + PBS (= Basal mixture) | |
| 7 | Diluted blood + PF4 (= mixture 2a) | |
| 8 | Diluted blood + PF4 + heparin 0.05 IU/ml | |
| | (= first mixture 2b′) | |
| 9 | Diluted blood + PF4 + heparin 0.5 IU/ml | |
| | (= second mixture 2b″) | |
| 10 | Diluted blood + PF4 + heparin 100 IU/ml | |
| | (= mixture 2c) | |

### 4/Fenestration Strategy (e.g. for Labeling CD62P):

First, on a first CD41-APC versus Count histogram, a threshold is positioned for distinguishing platelets (CD41+) from other blood cells that do not express the marker (CD41−):
[symbol] Platelet analysis

Then, among all CD41+ cells, the total median fluorescence intensity (MFI) of the CD62P marker is measured on a second histogram:
[symbol] Measurement of MFI of CD41+/CD62P cells

The threshold for distinguishing CD41+/CD62P− cells (non-activated platelets) from CD41+/CD62P+ double positive cells (activated platelets) is positioned at the intersection of the two peaks comprised between the activated condition with TRAP (tube 5) and the rest condition (tube 6) on the third CD62P-PE versus Count histogram. After repositioning the threshold on the second histogram, the percentage of CD41+/CD62P+ double positive cells is calculated for each condition tested:
[symbol] Measurement of the percentage of double positive CD41+/CD62P+ cells.

### 5/Expressions of Results:

According to one of the ways of calculation and of expression of the results as proposed in the above description, it is recalled that:
a/for each of the conditions, the results are expressed by multiplying the percentage of activated CD41+/CD62+ cells by the MFI (MFI CD62P) thereof. The product is denoted by “%×MFI” and expressed in arbitrary units (AU);
b/in order to dispense with variable platelet reactivity from one patient to another and from one manipulation to another, the raw results (%×MFI) obtained on the Validation/Standardization series standardize the expression of the results obtained on the Patient Test series by performing the following calculation for each tube:

$$A P A = 100 \times \frac{UA sample - UA Basal mixture}{UA max ( mixture 1 b ) - UA Basal mixture} \qquad [ Math 3 ]$$

c/the percentage inhibition of the TP series is also calculated with the formula:

$$\% T P series inhibition = 100 \times \frac{A P A max ( mixture 2 b ) - A P A mixture 2 c}{A P A max ( mixture 2 b )} \qquad [ Math 4 ]$$

6/Example of Results Obtained in Two Patients with Suspected HIT:

The slight differences that may be noted comprised between the following tables and the calculations given by the above formulas are inherent in the device used, which performs automatic value adjustments.

A/Results obtained with CD62P labeling:

The results obtained for the CD62P marker of patient 1 are as follows:

TABLE 3

| | | | | | | | Technical | | | |
|---|---|---|---|---|---|---|---|---|---|---|
| | | | | | | | Validation of | % | Interpretation | |
| | | | | | | | the VS series: | Ac-dependent | of the PT | |
| | | | | % | | % X | Activation | Platelet | Series: APA | |
| | | Heparin | | CD41+/ | MFI | MFI | ratio/Inhibition | Activation | maximum/% | |
| PATIENT 1 | Tube | (IU/ml) | PF4 | CD62P+ | CD62P | (UA) | ratio | (APA) | of inhibition | Conclusion |
| Standardization | 1 | 0 | + | 65% | 5592 | 3634.80 | 299.8 | | | VS series |
| Validation | 2 | 0.05 | + | 66% | 5606 | 3699.96 | | | | validated |
| Series | 3 | 0.5 | + | 70% | 6982 | 4887.40 | | | | |
| | 4 | 100 | + | 48% | 1272 | 610.56 | 0.88 | | | |
| Patient Trial | 5 | TRAP | − | 76% | 8214 | 6242.64 | | | | |
| Series | | (hep 0) | | | | | | | | |
| | 6 | 0 (basal) | − | 10% | 163 | 16.30 | | 0% | | Positive |
| | 7 | 0 | + | 47% | 1443 | 678.21 | | 14% | | PT series |
| | 8 | 0.05 | + | 54% | 2528 | 1365.12 | | 28% | 28% | |
| | 9 | 0.5 | + | 53% | 2414 | 1279.42 | | 26% | | |
| | 10 | 100 | + | 27% | 377 | 101.79 | | 2% | 94% | |

The results obtained for the CD62P marker of patient 2 areas follows:

TABLE 4

| | | | | | | | Technical | | | |
|---|---|---|---|---|---|---|---|---|---|---|
| | | | | | | | Validation of | % | Interpretation | |
| | | | | | | | the VS series: | Ac-dependent | of the PT | |
| | | | | % | | % X | Activation | Platelet | Series: APA | |
| | | Heparin | | CD41+/ | MFI | MFI | ratio/Inhibition | Activation | maximum/% | |
| PATIENT 2 | Tube | (IU/ml) | PF4 | CD62P+ | CD62P | (UA) | ratio | (APA) | of inhibition | Conclusion |
| Standardization | 1 | 0 | + | 15% | 201 | 30.15 | 13.2 | | | VS series |
| Validation | 2 | 0.05 | + | 22% | 280 | 61.60 | | | | validated |
| Series | 3 | 0.5 | + | 33% | 404 | 133.32 | | | | |
| | 4 | 100 | + | 8% | 165 | 13.20 | 0.90 | | | |
| Patient Trial | 5 | TRAP | − | 89% | 6487 | 5773.43 | | | | |
| Series | | (hep 0) | | | | | | | | |
| | 6 | 0 (basal) | − | 7% | 144 | 10.08 | | 0% | | Negative |
| | 7 | 0 | + | 7% | 156 | 10.92 | | 1% | | PT series |
| | 8 | 0.05 | + | 7% | 153 | 10.71 | | 1% | 1% | |
| | 9 | 0.5 | + | 6% | 149 | 8.94 | | −1% | | |
| | 10 | 100 | + | 6% | 149 | 8.94 | | −1% | * | |

b/Results obtained with Annexin V (AnxV) marker:

With the Annexin V labeling, a fenestration strategy and expression of results similar to the same presented for the CD62P protocol are applied.

The results obtained for the Annexin V marker for patient 1 are as follows:

TABLE 5

| | | | | | | | Technical | | | |
|---|---|---|---|---|---|---|---|---|---|---|
| | | | | | | | Validation of | % | Interpretation | |
| | | | | | | | the VS series: | Ac-dependent | of the PT | |
| | | | | % | | % X | Activation | Platelet | Series: APA | |
| | | Heparin | | CD41+/ | MFI | MFI | ratio/Inhibition | Activation | maximum/% | |
| PATIENT 1 | Tube | (IU/ml) | PF4 | AnxV+ | AnxV | (UA) | ratio | (APA) | of inhibition | Conclusion |
| Standardization | 1 | 0 | + | 31% | 537 | 166.47 | 95.9 | | | VS series |
| Validation | 2 | 0.05 | + | 31% | 474 | 146.94 | | | | validated |
| Series | 3 | 0.5 | + | 35% | 567 | 198.45 | | | | |
| | 4 | 100 | + | 12% | 247 | 29.64 | 0.85 | | | |
| Patient Test | 5 | Ca iono | − | 99% | 38310 | 37926.90 | | | | |
| series | | (hep 0) | | | | | | | | |
| | 6 | 0 (basal) | − | 3% | 69 | 2.07 | | 0% | | Positive |
| | 7 | 0 | + | 37% | 722 | 267.14 | | 135% | | PT series |
| | 8 | 0.05 | + | 36% | 625 | 225.00 | | 114% | 215% | |
| | 9 | 0.5 | + | 43% | 985 | 423.55 | | 215% | | |
| | 10 | 100 | + | 10% | 197 | 19.70 | | 9% | 96% | |

The results obtained for the Annexin V marker for patient 2 are as follows:

TABLE 6

| | | | | | | | Technical | | | |
|---|---|---|---|---|---|---|---|---|---|---|
| | | | | | | | Validation of | % | Interpretation | |
| | | | | | | | the VS series: | Ac-dependent | of the PT | |
| | | | | % | | % X | Activation | Platelet | Series: APA | |
| | | Heparin | | CD41+/ | MFI | MFI | ratio/Inhibition | Activation | maximum/% | |
| PATIENT 2 | Tube | (IU/ml) | PF4 | AnxV+ | AnxV | (UA) | ratio | (APA) | of inhibition | Conclusion |
| Standardization | 1 | 0 | + | 13% | 201 | 26.13 | 11.3 | | | VS series |
| Validation | 2 | 0.05 | + | 10% | 217 | 21.70 | | | | validated |
| Series | 3 | 0.5 | + | 10% | 193 | 19.30 | | | | |
| | 4 | 100 | + | 5% | 154 | 7.70 | 0.65 | | | |
| Patient Trial | 5 | Ca iono | − | 100% | 34310 | 34310.00 | | | | |
| series | | (hep 0) | | | | | | | | |
| | 6 | 0 (basal) | − | 3% | 64 | 1.92 | | 0% | | Negative |
| | 7 | 0 | + | 4% | 117 | 4.68 | | 14% | | PT series |
| | 8 | 0.05 | + | 4% | 147 | 5.88 | | 20% | 20% | |
| | 9 | 0.5 | + | 3% | 147 | 4.41 | | 13% | | |
| | 10 | 100 | + | 4% | 143 | 5.72 | | 19% | * | |

c/Interpretation of CD62P and Annexin V Results:

For the 2 patients, the Validation/Standardization (SV) series objectify an increase in the “%×MFI” parameter expressed in AU in the presence of PF4 and low concentrations of heparin (0.05 and/or 0.5 IU/ml, the tubes 2 and 3 respectively (mixtures 1b)) compared to the basal condition (PBS, the tube 6, Basal mixture), with inhibition of platelet activation in the presence of 100 IU/ml heparin (the tube 4, mixture 1c) for the 2 markers studied.

For the labeling of P-selectin (CD62P), a VS activation ratio of 299.8 and 13.2, for patients 1 and 2, respectively, demonstrates platelet activation in the presence of low concentrations of heparin (positivity threshold 5).

For the 2 patients, a VS inhibition ratio of 0.88 and 0.9, respectively, was obtained demonstrating an inhibition of the 5B9 antibody-induced activation in the presence of high concentration of heparin (threshold 0.5).

For the labeling of phosphatidylserine exposure with Annexin V, a VS activation ratio of 95.9 and 11.3 for patients 1 and 2, respectively, demonstrates platelet activation in the presence of low concentrations of heparin (positivity threshold 4). For the 2 patients, a ratio of inhibition of the VS series equal to 0.85 and 0.65, respectively, was obtained demonstrating an inhibition of activation induced by the antibody 5B9 in the presence of high concentration of heparin (threshold 0.5).

The results obtained with the VS series in the presence of the monoclonal antibody 5B9 demonstrate that the method according to the invention makes it possible to demonstrate platelet activation induced by the presence of anti-polyanion-modified PF4 activator antibodies in the blood of the patient tested and serves to interpret the results of the TP series.

For the labeling of the TP series CD62P expression in patient 1, the maximum antibody-dependent platelet activation (APA) calculated in the presence of low concentrations of heparin (0.05 and/or 0.5 IU/ml, mixtures 2b) (according to Math 3 described hereinabove) is 28% (positivity threshold 20%), with 94% inhibition (threshold 50%) in the presence of the high concentration of heparin tested (100 IU/ml, mixture 2c). For such parameter, the TP series is positive showing heparin-dependent platelet activation.

On the other hand, for patient 2, no platelet activation is objectified [in the] presence of low concentrations of heparin (0.05 and/or 0.5 IU/ml, mixtures 2b): maximum APA at 1% and thus below the positivity threshold of the test. For this parameter, the TP series is negative. The result of the functional test is not in favor of the diagnosis of HIT.

For labeling the phosphatidylserine exposure by TP series Annexin V in patient 1, the calculated antibody-dependent platelet activation percentage is 215% (positivity threshold ≥27%), with 96% inhibition (threshold 50%) in the presence of the high concentration of heparin tested (100 IU/ml, mixture 2c). For said parameter, the TP series is positive. The result of the functional test is in favor of the diagnosis of HIT.

On the other hand, for patient 2, no platelet activation is objectified in the presence of low concentrations of heparin (0.05 and/or 0.5 IU/ml, mixtures 2b): maximum APA at 16% and thus below the positivity threshold of the test. For this parameter, the TP series is negative. The result of the functional test is not in favor of the diagnosis of HIT.

The results (patients 1 and 2) were confirmed with the reference platelet activation test for the diagnosis of HIT: the Serotonin Release Assay (SRA).

As a reminder, the SRA is a functional test for the diagnosis of HIT. The assay is based on the measurement of the release, during platelet activation, of radiolabeled serotonin (¹⁴C-serotonin), previously incorporated into dense granules. Platelet washing steps serve to the elimination of the plasma matrix. The addition of different concentrations of heparin leads to the formation of PF4/H complexes at the surface of platelets of the healthy donor. The pathogenic antibodies recognize, by the Fab fragment thereof, the macromolecular PF4/H complexes and activate the platelets via the Fc fragment which binds to the FcγIIA receptor. The content of the granules is released into the reaction medium and the radioactivity measured in the supernatant is directly proportional to platelet activation.

### 7/Example of Results Obtained in Four Patients (Patients 3 to 6) Suspected of HIT:

The slight differences that may be noted comprised between the following tables and the calculations given by the above formulas are inherent in the device used, which performs automatic value adjustments.

A/Results obtained with CD62P labeling:

The results obtained for the CD62P marker of patient 3 are as follows:

TABLE 7

| | | | | | | | Technical | | | |
|---|---|---|---|---|---|---|---|---|---|---|
| | | | | | | | Validation of | % | Interpretation | |
| | | | | | | | the VS series: | Ac-dependent | of the PT | |
| | | | | % | | % X | Activation | Platelet | Series: APA | |
| | | Heparin | | CD41+/ | MFI | MFI | ratio/Inhibition | Activation | maximum/% | |
| PATIENT 3 | Tube | (IU/ml) | PF4 | CD62P+ | CD62P | (UA) | ratio | (APA) | of inhibition | Conclusion |
| Standardization | 1 | 0 | + | 54% | 802 | 433.08 | 94.2 | | | VS series |
| Validation | 2 | 0.05 | + | 54% | 749 | 404.46 | | | | validated |
| Series | 3 | 0.5 | + | 54% | 785 | 423.90 | | | | |
| | 4 | 100 | + | 44% | 415 | 182.60 | 0.57 | | | |
| Patient Trial | 5 | TRAP | − | 88% | 7409 | 6519.92 | | | | |
| series | | (hep 0) | | | | | | | | |
| | 6 | 0 (basal) | − | 5% | 90 | 4.50 | | 0% | | Positive |
| | 7 | 0 | + | 49% | 563 | 275.87 | | 65% | | PT series |
| | 8 | 0.05 | + | 50% | 604 | 302.00 | | 71% | 71% | |
| | 9 | 0.5 | + | 41% | 352 | 144.32 | | 33% | | |
| | 10 | 100 | + | 35% | 273 | 95.55 | | 22% | 69% | |

The results obtained for the D62P marker of patient 4 are as follows:

TABLE 8

| | | | | | | | Technical | | | |
|---|---|---|---|---|---|---|---|---|---|---|
| | | | | | | | Validation of | % | Interpretation | |
| | | | | | | | the VS series: | Ac-dependent | of the PT | |
| | | | | % | | % X | Activation | Platelet | Series: APA | |
| | | Heparin | | CD41+/ | MFI | MFI | ratio/Inhibition | Activation | maximum/% | |
| PATIENT 4 | Tube | (IU/ml) | PF4 | CD62P+ | CD62P | (UA) | ratio | (APA) | of inhibition | Conclusion |
| Standardization | 1 | 0 | + | 52% | 2152 | 1119.04 | 281.3 | | | VS series |
| Validation | 2 | 0.05 | + | 56% | 2618 | 1466.08 | | | | validated |
| Series | 3 | 0.5 | + | 68% | 5274 | 3586.32 | | | | |
| | 4 | 100 | + | 29% | 708 | 205.32 | 0.94 | | | |
| Patient Trial | 5 | TRAP | − | 91% | 9726 | 8850.66 | | | | |
| Series | | (hep 0) | | | | | | | | |
| | 6 | 0 (basal) | − | 5% | 255 | 12.75 | | 0% | | Negative |
| | 7 | 0 | + | 6% | 283 | 16.98 | | 0% | | PT series |
| | 8 | 0.05 | + | 8% | 299 | 23.92 | | 0% | 0% | |
| | 9 | 0.5 | + | 5% | 269 | 13.45 | | 0% | | |
| | 10 | 100 | + | 5% | 291 | 14.55 | | 0% | * | |

The results obtained for the 0D62P marker of patient 5 are as follows:

TABLE 9

| | | | | | | | Technical | | | |
|---|---|---|---|---|---|---|---|---|---|---|
| | | | | | | | Validation of | % | Interpretation | |
| | | | | | | | the VS series: | Ac-dependent | of the PT | |
| | | | | % | | % X | Activation | Platelet | Series: APA | |
| | | Heparin | | CD41+/ | MFI | MFI | ratio/Inhibition | Activation | maximum/% | |
| PATIENT 5 | Tube | (IU/ml) | PF4 | CD62P+ | CD62P | (UA) | ratio | (APA) | of inhibition | Conclusion |
| Standardization | 1 | 0 | + | 46% | 1400 | 644.00 | 52.7 | | | VS series |
| Validation | 2 | 0.05 | + | 46% | 1386 | 637.56 | | | | validated |
| Series | 3 | 0.5 | + | 52% | 2361 | 1227.72 | | | | |
| | 4 | 100 | + | 32% | 598 | 191.36 | 0.84 | | | |
| Patient Test | 5 | TRAP | − | 71% | 7308 | 5188.68 | | | | |
| series | | (hep 0) | | | | | | | | |
| | 6 | 0 (basal) | − | 10% | 233 | 23.30 | | 0% | | Positive |
| | 7 | 0 | + | 41% | 989 | 405.49 | | 32% | | PT series |
| | 8 | 0.05 | + | 40% | 946 | 378.40 | | 29% | 35% | |
| | 9 | 0.5 | + | 42% | 1060 | 445.20 | | 35% | | |
| | 10 | 100 | + | 25% | 474 | 118.50 | | 8% | 77% | |

The results obtained for the 0D62P marker of patient 6 are as follows:

TABLE 10

| | | | | | | | Technical | | | |
|---|---|---|---|---|---|---|---|---|---|---|
| | | | | | | | Validation of | % | Interpretation | |
| | | | | | | | the VS series: | Ac-dependent | of the PT | |
| | | | | % | | % X | Activation | Platelet | Series: APA | |
| | | Heparin | | CD41+/ | MFI | MFI | ratio/Inhibition | Activation | maximum/% | |
| PATIENT 6 | Tube | (IU/ml) | PF4 | CD62P+ | CD62P | (UA) | ratio | (APA) | of inhibition | Conclusion |
| Standardization | 1 | 0 | + | 42% | 1013 | 425.46 | 117.1 | | | VS series |
| Validation | 2 | 0.05 | + | 44% | 1125 | 495.00 | | | | validated |
| Series | 3 | 0.5 | + | 55% | 1842 | 1013.10 | | | | |
| | 4 | 100 | + | 18% | 359 | 64.62 | 0.94 | | | |
| Patient Test | 5 | TRAP | − | 92% | 7273 | 6691.16 | | | | |
| series | | (hep 0) | | | | | | | | |
| | 6 | 0 (basal) | − | 5% | 173 | 8.65 | | 0% | | Negative |
| | 7 | 0 | + | 13% | 268 | 34.84 | | 3% | | PT series |
| | 8 | 0.05 | + | 17% | 331 | 56.27 | | 5% | 5% | |
| | 9 | 0.5 | + | 8% | 211 | 16.88 | | 1% | | |
| | 10 | 100 | + | 6% | 204 | 12.24 | | 0% | * | |

B/Results Obtained with the Annexin V Marker:

The results obtained for the AnxV marker of patient 3 are as follows:

TABLE 11

| | | | | | | | Technical | | | |
|---|---|---|---|---|---|---|---|---|---|---|
| | | | | | | | Validation of | % | Interpretation | |
| | | | | | | | the VS series: | Ac-dependent | of the PT | |
| | | | | % | | % X | Activation | Platelet | Series: APA | |
| | | Heparin | | CD41+/ | MFI | MFI | ratio/Inhibition | Activation | maximum/% | |
| PATIENT 3 | Tube | (IU/ml) | PF4 | AnxV+ | AnxV | (UA) | ratio | (APA) | of inhibition | Conclusion |
| Standardization | 1 | 0 | + | 16% | 263 | 42.08 | 21.7 | | | VS series |
| Validation | 2 | 0.05 | + | 17% | 271 | 46.07 | | | | validated |
| Series | 3 | 0.5 | + | 18% | 286 | 51.48 | | | | |
| | 4 | 100 | + | 7% | 193 | 13.51 | 0.74 | | | |
| Patient Test | 5 | Ca iono | − | 97% | 45498 | 44133.06 | | | | |
| series | | (hep 0) | | | | | | | | |
| | 6 | 0 (basal) | − | 3% | 79 | 2.37 | | 0% | | Positive |
| | 7 | 0 | + | 19% | 283 | 53.77 | | 105% | | PT series |
| | 8 | 0.05 | + | 17% | 283 | 48.11 | | 93% | 93% | |
| | 9 | 0.5 | + | 17% | 273 | 46.41 | | 90% | | |
| | 10 | 100 | + | 6% | 192 | 11.52 | | 19% | 80% | |

The results obtained for the AnxV marker of patient 4 are as follows:

TABLE 12

| | | | | | | | Technical | | | |
|---|---|---|---|---|---|---|---|---|---|---|
| | | | | | | | Validation of | % | Interpretation | |
| | | | | | | | the VS series: | Ac-dependent | of the PT | |
| | | | | % | | % X | Activation | Platelet | Series: APA | |
| | | Heparin | | CD41+/ | MFI | MFI | ratio/Inhibition | Activation | maximum/% | |
| PATIENT 4 | Tube | (IU/ml) | PF4 | AnxV+ | AnxV | (UA) | ratio | (APA) | of inhibition | Conclusion |
| Standardization | 1 | 0 | + | 40% | 1837 | 734.80 | 411.7 | | | VS series |
| Validation | 2 | 0.05 | + | 32% | 624 | 199.68 | | | | validated |
| Series | 3 | 0.5 | + | 46% | 2721 | 1251.66 | | | | |
| | 4 | 100 | + | 15% | 392 | 58.80 | 0.95 | | | |
| Patient Test | 5 | Ca iono | − | 99% | 38047 | 37666.53 | | | | |
| series | | (hep 0) | | | | | | | | |
| | 6 | 0 (basal) | − | 4% | 76 | 3.04 | | 0% | | Negative |
| | 7 | 0 | + | 5% | 135 | 6.75 | | 0% | | PT series |
| | 8 | 0.05 | + | 8% | 176 | 14.08 | | 1% | 1% | |
| | 9 | 0.5 | + | 5% | 212 | 10.60 | | 1% | | |
| | 10 | 100 | + | 8% | 256 | 20.48 | | 1% | * | |

The results obtained for the AnxV marker of patient 5 are as follows:

TABLE 13

| | | | | | | | Technical | | | |
|---|---|---|---|---|---|---|---|---|---|---|
| | | | | | | | Validation of | % | Interpretation | |
| | | | | | | | the VS series: | Ac-dependent | of the PT | |
| | | | | % | | % X | Activation | Platelet | Series: APA | |
| | | Heparin | | CD41+/ | MFI | MFI | ratio/Inhibition | Activation | maximum/% | |
| PATIENT 5 | Tube | (IU/ml) | PF4 | AnxV+ | AnxV | (UA) | ratio | (APA) | of inhibition | Conclusion |
| Standardization | 1 | 0 | + | 33% | 572 | 188.76 | 8.6 | | | VS series |
| Validation | 2 | 0.05 | + | 35% | 710 | 250.10 | | | | validated |
| Series | 3 | 0.5 | + | 38% | 860 | 328.67 | | | | |
| | 4 | 100 | + | 26% | 497 | 126.74 | 0.61 | | | |
| Patient Test | 5 | Ca iono | − | 98% | 17737 | 17387.09 | | | | |
| series | | (hep 0) | | | | | | | | |
| | 6 | 0 (basal) | − | 19% | 197 | 38.34 | | 0% | | Positive |
| | 7 | 0 | + | 40% | 983 | 396.34 | | 123% | | PT series |
| | 8 | 0.05 | + | 38% | 880 | 335.71 | | 102% | 169% | |
| | 9 | 0.5 | + | 43% | 1233 | 528.83 | | 169% | | |
| | 10 | 100 | + | 25% | 463 | 114.36 | | 26% | 85% | |

The results obtained for the AnxV marker of patient 6 are as follows:

TABLE 14

| | | | | | | | Technical | | | |
|---|---|---|---|---|---|---|---|---|---|---|
| | | | | | | | Validation of | % | Interpretation | |
| | | | | | | | the VS series: | Ac-dependent | of the PT | |
| | | | | % | | % X | Activation | Platelet | Series: APA | |
| | | Heparin | | CD41+/ | MFI | MFI | ratio/Inhibition | Activation | maximum/% | |
| PATIENT 6 | Tube | (IU/ml) | PF4 | AnxV+ | AnxV | (UA) | ratio | (APA) | of inhibition | Conclusion |
| Standardization | 1 | 0 | + | 17% | 229 | 38.93 | 26.5 | | | VS series |
| Validation | 2 | 0.05 | + | 17% | 219 | 37.23 | | | | validated |
| Series | 3 | 0.5 | + | 20% | 258 | 51.60 | | | | |
| | 4 | 100 | + | 9% | 143 | 12.87 | 0.75 | | | |
| Patient Test | 5 | Ca iono | − | 98% | 27925 | 27366.50 | | | | |
| series | | (hep 0) | | | | | | | | |
| | 6 | 0 (basal) | − | 3% | 65 | 1.95 | | 0% | | Negative |
| | 7 | 0 | + | 7% | 127 | 8.89 | | 14% | | PT series |
| | 8 | 0.05 | + | 7% | 134 | 9.38 | | 15% | 15% | |
| | 9 | 0.5 | + | 7% | 130 | 9.10 | | 14% | | |
| | 10 | 100 | + | 7% | 118 | 8.26 | | 13% | * | |

c/Interpretation of CD62P and Annexin V Results:

For patients 3 to 6, the VS series objectify:
an increase in the parameter “%×MFI” expressed as AU in the presence of PF4 and in the presence of low concentrations of heparin (0.05 and/or 0.5 IU/ml, the tubes 2 and/or 3 respectively, the mixtures 1b) compared to the basal condition (PBS, the tube 6, the Basal mixture) due to the activation ratio of the VS series above the threshold,
and an inhibition of platelet activation in the presence of 100 IU/ml heparin (the tube 4, the mixture 1c) for the 2 markers studied with the inhibition ratio of the VS series above the threshold. The inhibition of platelet activation in the presence of high concentration of heparin is characteristic of HIT antibodies.

The results obtained with the VS series in the presence of the monoclonal antibody 5B9 demonstrate that the method according to the invention serves to demonstrate platelet activation induced by the presence of anti-PF4/H activator antibodies in the blood of the patient tested.

With regard to the results obtained for the TP series with the blood of patients 3 and 5, an increase in the APA is observed in the presence of low concentrations of heparin (0.05 and/or 0.5 IU/ml, the tube(s) 8 and/or 9 respectively, i.e. the mixture(s) 2b) compared to the basal condition (the tube 6, PBS, The Basal mixture). The inhibition of platelet activation in the presence of 100 IU/ml heparin (the tube 10, the mixture 2c) is observed for the 2 markers studied. The results are a proof of to the presence of activator (pathogen) and heparin-dependent anti-PF4 antibodies in the sample. The results obtained for patients 3 and 5 are in favor of the diagnosis of HIT. The results of the biological tests should be compared with the clinical data in order to confirm or exclude the diagnosis of heparin-induced thrombocytopenia.

On the other hand, for patients 4 and 6, no platelet activation is objectified, regardless of the marker used, which demonstrates the absence of activator antibodies (pathogens) in the blood of said patients. The results obtained for patients 4 and 6 are not in favor of the diagnosis of HIT.

The results (patients 3 to 6) were confirmed with the reference assay (SRA, Serotonine Release Assay).

1. An analysis method for analyzing heparin-dependent platelet activation in a patient suspected of having thrombocytopenia induced by polyanion-modified antiplatelet factor 4 (PF4) antibodies, comprising the following steps:
1) the preparation of a series of tests necessary for validation and standardization, hereinafter referred to as the Validation/Standardization series (VS), said Validation/Standardization series comprising:
mixing a sample of whole blood from the patient, diluted in an acceptable buffer, in the presence of exogenous platelet factor (PF4), and a monoclonal antibody specifically directed against the platelet factor 4 modified by a polyanion, preferably by heparin, or one of the fragments thereof, in order to obtain a mixture 1a;
at least one mixture of a sample of whole blood from said patient, diluted in an acceptable buffer, with exogenous PF4, a monoclonal antibody specifically directed against polyanion-modified PF4, or a fragment thereof, and heparin in a concentration comprised between 0.01 and 1 IU/ml, in order to obtain at least one mixture 1b;
at least one mixture of a sample of whole blood from said patient, diluted in an acceptable buffer, with exogenous PF4, a monoclonal antibody specifically directed against polyanion-modified PF4, or a fragment thereof, and heparin in a concentration comprised between 10 and 200 IU/ml, in order to obtain at least one mixture 1c;
2) the preparation of a Patient Test (PT) series, said Patient Test series comprising:
mixing a sample of whole blood from said patient, diluted in an acceptable buffer, with a platelet activator or a strong agonist, to obtain a mixture Ago;
mixing a sample of whole blood from said patient, diluted in an acceptable buffer, to obtain the mixture Basal;
mixing a sample of whole blood from said patient, diluted in an acceptable buffer, with PF4 to obtain a mixture 2a;
at least one mixing of a sample of whole blood from said patient, diluted in an acceptable buffer, with PF4 and heparin at a concentration equal to the concentration used for mixture 1b, in order to obtain a mixture 2b;
at least one mixing of a sample of whole blood from said patient, diluted in an acceptable buffer, with PF4 and heparin at a concentration equal to the concentration used for mixture 1c, in order to obtain a mixture 2c;
3) the incubation of all mixtures of the VS and TP series and the subsequent labeling of the platelet population and the specific labeling of activated platelets in said population in each mixture; and
4) the analysis of all mixtures obtained in step 3).

2. The analysis method according to claim 1, wherein the patient is suspected of having thrombocytopenia induced by antibodies to heparin-modified PF4.

3. The method according to claim 1 comprising the preparation of at least two mixtures 1b in step 1), preferably two mixtures 1b (i.e. the mixtures 1b′ and 1b″) are carried out on the one hand with a concentration of heparin comprised between 0.01 and 0.1 IU/ml, preferably between 0.02 and 0.08 IU/ml, for the first mixture 1b′, and on the other hand comprised between 0.2 and 1 IU/ml, preferably between 0.3 and 0.8 IU/ml for the second mixture 1b″.

4. The method according to claim 1, comprising, following step 4), the additional step 5) of validation of the VS series by the calculation of the activation ratio of the series and the calculation of the inhibition ratio of the same series.

5. The method according to claim 4, wherein the VS series is validated if one observes:
an activation of platelets in the sample of said series induced by an antibody or antibody fragment directed specifically against the polyanion-modified PF4, in the absence of heparin and/or in the presence of low concentrations of heparin, and
an inhibition of the activation in the presence of high concentrations of heparin.

6. The method according to claim 1, which is a flow cytometry analysis method.

7. The method according to claim 4, comprising, following step 5), a step 5′) of calculating the antibody-dependent platelet activation level (called APA) of the mixtures Ago, 2a, 2b and 2c of the TP series.

8. The method according to claim 1, wherein all the samples of whole blood of the patient used are diluted to 1/10, preferably diluted to 1/2 to 1/10^{th} and preferably to a quarter, in physiological saline.

9. The method according to claim 1, wherein the monoclonal antibody specifically directed against polyanion-modified PF4 comprises:
as VH variable region, the amino acid sequence encoded by SEQ ID NO:2, and
as VL variable region, the amino acid sequence encoded by SEQ ID NO:1.

10. The method according to claim 1, wherein the platelet labeling of step 3) is carried out using at least one marker specific for the platelet population and at least one marker specific for the state of activation of the platelets, preferably using at least one marker specific to the platelet population and possibly two markers specific to state of activation of the platelets.

11. The method according to claim 10, wherein the platelet labeling of step 3) is performed using at least one platelet population-specific marker selected from the CD41 marker, the CD61 marker and combination of the CD41/CD61 markers, and at least one marker specific to the state of activation of the platelets, preferably two, chosen from P-selectin and phosphatidylserine.

12. A method of diagnosis of the exclusion of heparin-induced thrombocytopenia (HIT) in a patient, comprising the method according to claim 7, as well as a step 6) of interpretation of the calculated antibody-dependent platelet activation (APA) rates in each mixture of the TP series for the diagnosis of HIT exclusion.

13. The method according to claim 12, wherein a diagnosis of HIT exclusion is made when no platelet activation is observed in the presence of low concentrations of heparin on the patient's TP series.

14. A method of confirmation of a diagnosis of HIT in a patient suspected of having the illness, comprising the method according to claim 7, as well as a step 6) of interpretation of the calculated antibody-dependent platelet activation (APA) rates in each mixture of the TP series for the diagnosis of HIT.

15. The method according to claim 14, wherein a diagnosis of HIT in a patient suspected of having the illness is confirmed when platelet activation of the TP series of the patient is observed in the presence of low concentrations of heparin, and an inhibition of platelet activation in the presence of high concentrations of heparin.
